(19) United States
(12) Patent Application Publication
(10) Pub. No.: US 2025/0256042 A1
(43) Pub. Date: Aug. 14, 2025

(54) ACCESSORY FOR INJECTION DEVICE

(71) Applicant: JANSSEN BIOTECH, INC., Horsham, PA (US)

(72) Inventors: EDWARD KERNICK, Jacksonville, FL (US); Vincent BARRE, Jacksonville, FL (US); Dominic GOURD, Ponte Vedra, FL (US); Kathy BROWN, Vacaville, CA (US)

(21) Appl. No.: 19/099,999

(22) PCT Filed: Aug. 17, 2023
(86) PCT No.: PCT/IB2023/058257

§ 371 Date: Jan. 30, 2025

(51) Int. Cl. A61M 5/42

(52) U.S. Cl. CPC A61M 5/427

(57) ABSTRACT

In one example, an accessory for a drug injection device has a proximal end, a distal end, a receptacle, and a base. The receptacle defines a cavity that extends into the proximal end towards the distal end and terminates at an inner surface. The cavity receives an injection end of the drug injection device therein so as to position a needle of the drug injection device for delivery of a medication. The receptacle defines an opening that extends into the distal end and through the inner surface. The opening receives the needle of the drug injection device therethrough and has a cross-sectional dimension that is smaller than a cross-sectional dimension of the cavity. The base is disposed at the distal end of the injection device and has a skin-contacting surface with a cross-sectional dimension that is greater than the cross-sectional dimension of the cavity.

### TECHNICAL FIELD

The present disclosure relates to drug injection devices, and more specifically, but not necessarily exclusively, to accessories for drug injection devices.

### BACKGROUND

A drug injection device, such as (without limitation) a syringe, a manual injector, a pen injector, and/or an autoinjector, is commonly used to inject a liquid drug into a patient. These devices often include a plunger rod, a drug container, and a needle. The drug container can be, for example, a syringe body, a cartridge, or a vial. In operation, the drug injection device is pressed against a patient's skin such that the needle penetrates the patient's skin by a desired depth. The plunger rod is then driven by a manual force (i.e., in the case of a syringe or manual injector) or by an automated force (i.e., in the case of an autoinjector) to drive the drug product from the drug container and out of the needle into the patient. The automated force can be the force of a spring, motor, or other suitable energy source.

Some drug injection devices have a retractable sleeve that is configured to move between an extended position and a retracted position. In some such devices, the retractable sleeve can unlock an actuation mechanism of the drug injection device when it is moved to the retracted position. In other such devices, the retractable sleeve can act as a needle guard that extends over the needle to prevent inadvertent needle pricks, and moves to the retracted position to expose the needle for injection. In either case, the retractable sleeve is placed into engagement with the patient's skin, and the drug injection device is forced towards the patient's skin thereby causing the retractable sleeve to move to retract relative to the injection device housing from the extended position to the retracted position.

### BRIEF DESCRIPTION OF THE DRAWINGS

The following description of the illustrative embodiments may be better understood when read in conjunction with the appended drawings. It is understood that potential embodiments of the disclosed systems and methods are not limited to those depicted.

FIG. 1 shows a perspective view of a drug injection device according to one example;

FIG. 2 shows a side view of the drug injection device of FIG. 1 with a portion of the housing removed;

FIG. 3 shows a side view of the drug injection device of FIG. 1 with an entirety of the housing removed;

FIG. 4 shows a perspective view of an accessory for a drug injection device according to one example, with hidden lines shown;

FIG. 5 shows a perspective view of an accessory for a drug injection device according to another example;

FIG. 6 shows a side view of a drug injection device and the accessory of FIG. 5 in position against a patient's skin for an injection;

FIG. 7 shows a side view of an accessory for a drug injection device according to yet another example;

FIG. 8 shows a perspective view of an accessory for a drug injection device according to yet still another example;

FIG. 9 shows a perspective view of the accessory of FIG. 8 with a drug injection device;

FIG. 10 shows the accessory of FIGS. 7 and 8 positioned against a patient's skin with a drug injection device;

FIG. 11 shows a perspective view of an accessory for a drug injection device according to another example; and

FIG. 12 shows a perspective view of an accessory for a drug injection device according to yet another example.

### DETAILED DESCRIPTION OF ILLUSTRATIVE EMBODIMENTS

When using a drug injection device having a sliding sleeve, it is preferable that the drug injection device is pressed into the patient's skin with only as much force as needed to retract the sliding sleeve. However, it has been observed that, in practice, operators of the drug injection device often press the device into the patient's skin with an excessive force that is greater than that needed to retract the sliding sleeve. This excessive force can result in the patient's skin being compressed toward the patient's bone beneath the injection site, thereby reducing the distance between the drug injection device and the patient's bone. In extreme instances, the patient's skin can be compressed to such a degree that the tip of the needle can come into contact with the patient's bone, which can bend the needle under the patient's skin. Moreover, as the skin is compressed at the injection site, uncompressed skin adjacent the injection site can surround and collapse towards the sliding sleeve, thereby making it difficult to visually determine when the sliding sleeve has reached the retracted position. This can exacerbate the issue, as the patient or health care provider could continue to press the drug injection device further into the skin, without knowing that the sliding sleeve has reached the retracted position.

It has also been observed that, due to user variability, the location that an injection is administered often varies from one injection to the next for a given patient and varies from one patient to the next. This variability can result in injections being administered at different locations with differing amounts of soft tissue (i.e., fat and/or muscle). However, the likelihood of the needle contacting bone is higher when injections are administered at locations having smaller amounts of soft tissue. Therefore, it is often desirable to administer injections at locations having higher amounts of soft tissue.

The present disclosure relates to accessories that can be paired with drug injection devices to address the aforementioned and/or other issues, and also relates to drug injection systems that include such accessories and drug injection devices. It will be understood that, according to various examples, accessories of the present disclosure can be paired with different drug injection devices, such as (without limitation) syringes, manual injectors, pen injectors, and/or autoinjectors. However, for ease of discussion, one example of a drug injection device 110 will be described herein.

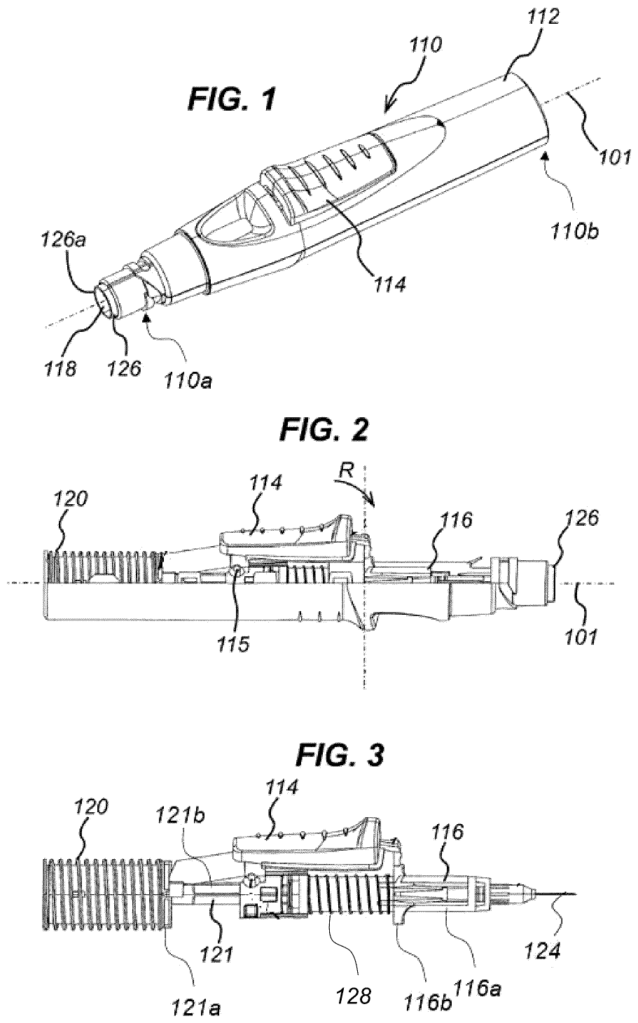

Referring to FIGS. 1 to 3, a drug injection device 110 is shown according to one example. In this example, the drug injection device 110 is an autoinjector. The drug injection device 110 comprises a housing 112, and a syringe 116 housed within the housing. The syringe 116 comprises a syringe body 116a having a needle 124 at one end and a flange 116b at the other end. The drug injection device 110 comprises a sliding sleeve 126 that is configured to move relative to the housing 112 between an extended position, wherein the sliding sleeve 126 extends from the housing 112, and a retracted position, wherein the sliding sleeve 126 is retracted relative to the housing 112. Retraction of the sliding sleeve 126 can be caused by applying a force to the device 110 to move the sliding sleeve 126 against the skin of the patient. Retraction of the sliding sleeve 126 unlocks a drive mechanism of the drug injection device 110, thereby allowing the injection to begin. As will be described further below, when the injection is initiated, the syringe 116, including the syringe body 116a and needle 124, is advanced from an initial position, wherein the needle 124 does not extend from an opening 118 at a first end 110a of the drug injection device 110, to an injection position, where the needle 124 projects out of the opening 118 at a first end 110a. When the injection is complete, the syringe 116 is advanced proximally to a final position, wherein the needle 124 is retracted back into the opening 118.

The drug injection device 110 can have a pen-like shape. For instance, the drug injection device 110 can have a first end 110a (also referred to as an injection end) and a second end 110b. The first end 110a and second end 110b can be opposite one another along a central axis 101. The first end 110a can include the sliding sleeve 126 and a distal end of the housing 112. The drug injection device 110 can be elongate from the first end 110a to the second end 110b. The housing 112 can have a substantially tubular shape, although other shapes are contemplated within the scope of this disclosure. The sliding sleeve 126 can be configured to extend from, and retract into, a distal aperture 118 of the housing 112.

The drug injection device 110 comprises a drive element 121 in lieu of a conventional plunger that would normally be used to discharge the contents of the syringe 116 manually. The drive element 121 has a drive surface 121a, and a plunger having a plunger rod 121b and a seal (e.g., bung). The seal is configured to form a seal with an inner wall of the syringe body 116a so as to prevent leakage of the drug past the seal.

The drug injection device 110 comprises a drive spring 120 that, when released, engages the drive surface 121a to drive the drive element 121 distally. Hydrostatic forces acting through the drug and/or, to some extent, static friction between the drive element 121 and the syringe body 116a initially ensure that the plunger advances with the syringe body 116a so as to move the syringe 116 from the initial position, whereby the needle 124 does not extend from the opening 124, to the injection position, whereby the needle 124 extends from the opening 118. The plunger advances with the syringe body 116a until the syringe body 116a meets an obstruction that retards its motion or a return spring 128 bottoms out. At this point, the syringe 116 is in the injection position and the drive spring 120 continues to drive the plunger so that it advances inside the syringe body 116a to dispense the drug from the needle 124. After the drug has been dispensed, the return spring 128 acts on the syringe 116 to return the syringe 116 from the injection position to a final position, wherein the needle 124 is retracted into the opening 118 of the housing 112.

The drug injection device 110 can comprise an actuator 114 that is configured to be actuated to release the drive spring 120. The actuator 114 can be configured to move between an unactuated position, wherein the drive spring 120 is not released, and an actuated position, wherein the drive spring 120 is released. The drug injection device 110 can be configured such that, movement of the sliding sleeve 126 from the extended position to the retracted position can unlock the actuator 114, thereby allowing the actuator to move from the unactuated position to the actuated position to deliver the injection.

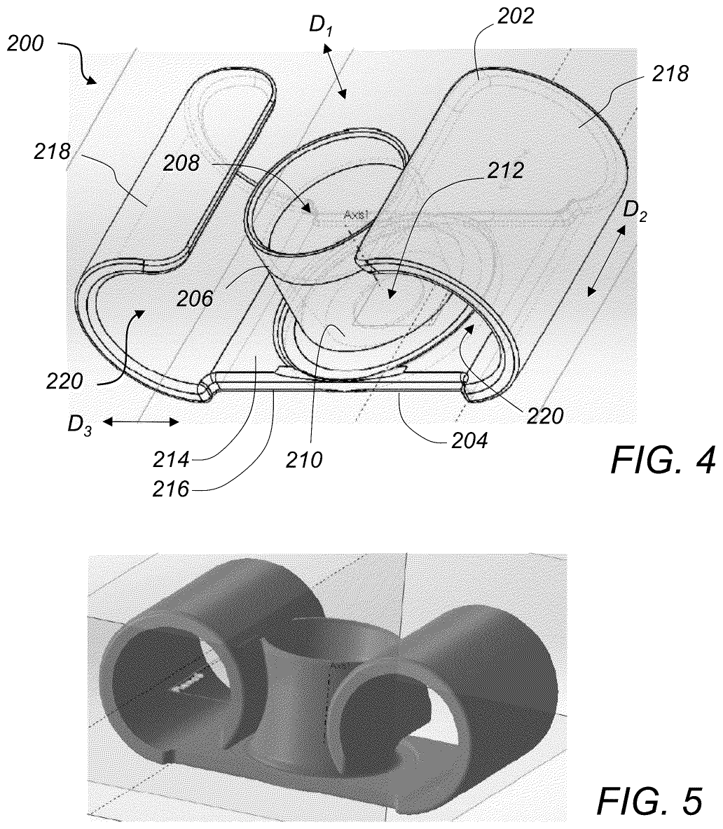

Turning now to FIG. 4, an accessory 200 for a drug injection device, such as device 110 or any other suitable drug injection device, is shown according to one example. The accessory 200 has a proximal end 202 and a distal end 204 that are offset from one another along a first direction D1. The accessory 200 has a receptacle 206 defining a cavity 208 that extends into the proximal end 202 towards the distal end 204. The cavity 208 is configured to receive the injection end 110a of the drug injection device 110 therein. When received in the cavity 208, the injection end 110a can be removably coupled to the receptacle 206. The drug injection device 110 and the receptacle can removably couple to one another such that the accessory does not cause the sliding sleeve 126 to be locked in the retracted position. Rather, the sliding sleeve 126 can return to the extended position merely by the operator removing the force from the drug injection device that is applied towards the patient.

The cavity 208 can terminate at an inner surface 210. In at least some examples, the inner surface 210 can limit an insertion depth of the drug injection device 110 into the cavity 208. The cavity 208 can be configured to conform to a shape and size of the injection end 110a of the drug injection device 110. For example, the cavity 208 can have a cross-sectional shape that conforms to a cross-sectional shape of the injection end 110a. The cavity 208 can have a cross-sectional dimension that is substantially equal to a cross-sectional dimension of the injection end 110a. As such, the cavity 208 can be configured to position the injection device 110 relative to the accessory 200 by limiting, or substantially preventing altogether, movement of the injection device 110 along a second direction D2 and/or a third direction D3, wherein the first, second, and third directions D1, D2, and D3 are perpendicular to one another.

The receptacle 206 can define an opening 212 (shown in hidden lines) that extends into the distal end 204 and through the inner surface 210. The opening 212 is configured to receive the needle 124 of the drug injection device 110 therethrough. The opening 212 has a cross-sectional dimension that is smaller than a cross-sectional dimension of the cavity 208. For example, a diameter or width of the opening 212 along a direction perpendicular to the first direction DI can be smaller than a diameter or width of the cavity 208 along the same direction. As such, the inner surface 210 can interfere with the sliding sleeve 126 and/or distal end of the housing 112 of the drug injection device 110 to limit the insertion depth of the drug injection device 110 into the accessory 200.

The accessory 200 has a base 214 at the distal end 204. The base 214 has a skin-contacting surface 216 that has a cross-sectional dimension and/or surface area that is greater than a cross-sectional dimension and/or area, respectively, of the cavity 208. For example, the skin-contacting surface 216 can be wider than the cavity 208 in at least one direction, such as the second direction D2 and/or third direction D3. The base 214 can be configured as a flange. Moreover, the skin-contacting surface 216 can have a cross-sectional dimension and/or surface area that is greater than the cross-sectional dimension and/or surface area, respectively, of a skin-contacting surface 126a at the injection end 110a of the drug injection device 110. For example, the skin-contacting surface 216 can be wider than the cavity 208 in at least one direction, such as the second direction D2 and/or third direction D3. Thus, when the injection end 110a of the drug injection device 110 is received in the cavity 208, the skin-contacting surface 216 of the base 214 can extend outwardly from the injection end 110a along a direction along a plane that is transverse to the first direction D1.

The accessory 200 can have at least one finger holder 218. The at least one finger holder 218 can define an opening 220 therein that is sized to receive a finger of an operator of the drug injection device 110. The opening 220 can extend in a direction that is transverse to the first direction D1. For example, the opening 220 can extend along an axis that extends along the second direction D2. In some examples, the at least one finger holder 218 can define an at least partially closed shape around the opening 220 as shown in FIG. 4 or more of a closed shape as shown in FIG. 5. The at least one finger holder 218 can extend from the base 214 along the first direction D1 and then along a direction (e.g., the third direction D3) that is towards the receptacle 206 as shown in FIG. 4. In some examples, the at least one finger holder 218 can further extend back towards the base 214 as shown in FIG. 5.

In some examples, as shown, the accessory 200 can have a pair of finger holders 218. In such examples, the finger holders 218 can be disposed on opposite sides of the receptacle 206. The finger holders 218 can be substantial mirror images of one another or can differ from one another.

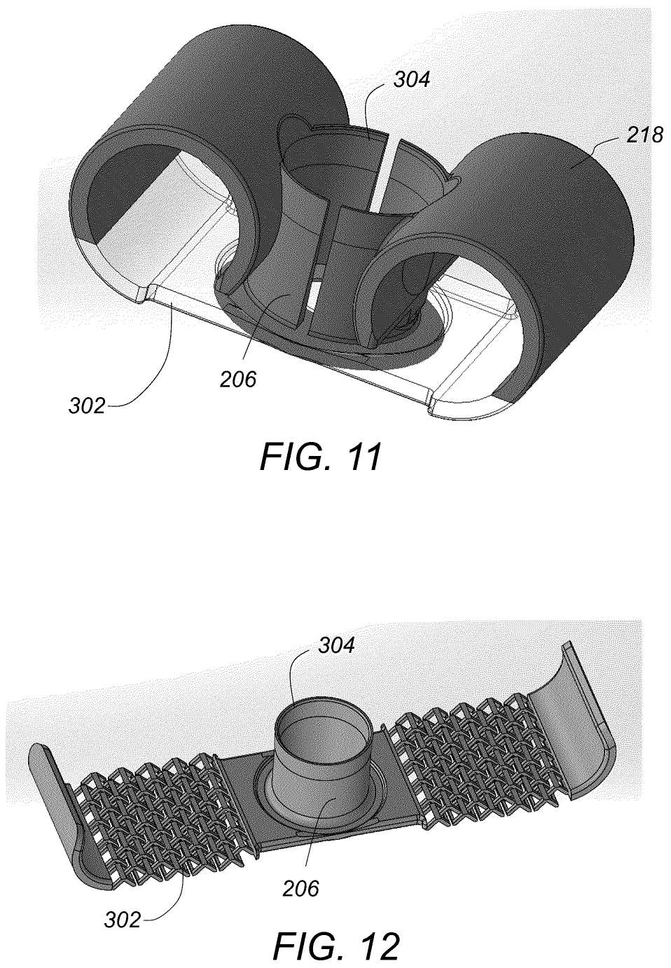

In some examples, as shown in FIGS. 11 and 12, a first portion 302 that includes at least a portion of the skin contacting surface and a second portion 304 that supports the drug injection device 110. The second portion 304 can include the receptacle 206 and/or at least a portion of the finger holders 218. The first portion 302 can be more flexible than the second portion 304 so as to allow a user to flex the accessory to pinch the patient's skin while the accessory is positioned on the patient's skin during an injection to limit risk of advertent needle sticks. For example, as shown in FIG. 11, the first portion 302 can be formed of a material (e.g., rubber and/or silicone) that is more flexible than a material (e.g., plastic and/or metal) of the second portion 304. As another example, as shown in FIG. 12, the first portion 302 can be formed of the same material as the second portion 304 but can be formed in a manner to have a greater flexibility than the second portion 304. In this example, the first portion 302 is formed to have a chainmail-like structure. However, the first portion 302 can be formed in another manner to be more flexible than the second portion 304 (e.g., to have another structure that is more flexible).

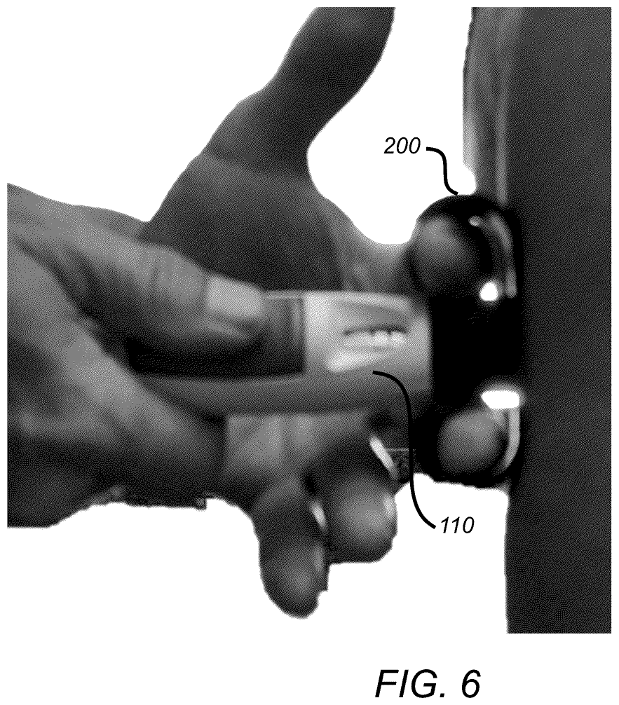

Turning briefly to FIG. 6 (and with reference to labels in FIGS. 4 and 5), in operation, the accessory 200 can be positioned against the skin of the patient at an injection site, such as a back of the arm. The injection end 110a of the drug injection device 110 can be received into the receptacle 206 before positioning the accessory 200, which positioning the accessory 200, or after positioning the accessory against the skin. The drug injection device 110 can then be forced towards the skin, thereby causing the sliding sleeve 126 to engage the inner surface 210 of the receptacle 206 and retract. This in turn exposes the needle 124 and causes the needle 124 to be inserted into the patient's skin. At least a portion of the force applied to the drug injection device 110 can be transferred to the patient's skin through the accessory 200, thereby causing at least some deflection of the skin. The drug injection device 110 can then be activated (e.g., by automatic or manual force) to cause the drug to be discharged from the drug container 116 and out of the needle 124.

When the force is applied to move the drug injection device 110 towards the patient, the skin-contacting surface 216 of the accessory 200 can spread out the force over an area of the patient's skin that is greater than the surface area of the skin-contacting surface 126a of the sliding sleeve 126. Spreading out the force over a greater area can result in a smaller deflection of the skin towards the bone compared to the deflection that would occur without the use of the accessory. In other words, an application of a force to the accessory 200 can result in a smaller skin deflection towards the bone than an application of an equivalent force to the drug injection device 110 when used without the accessory 200.

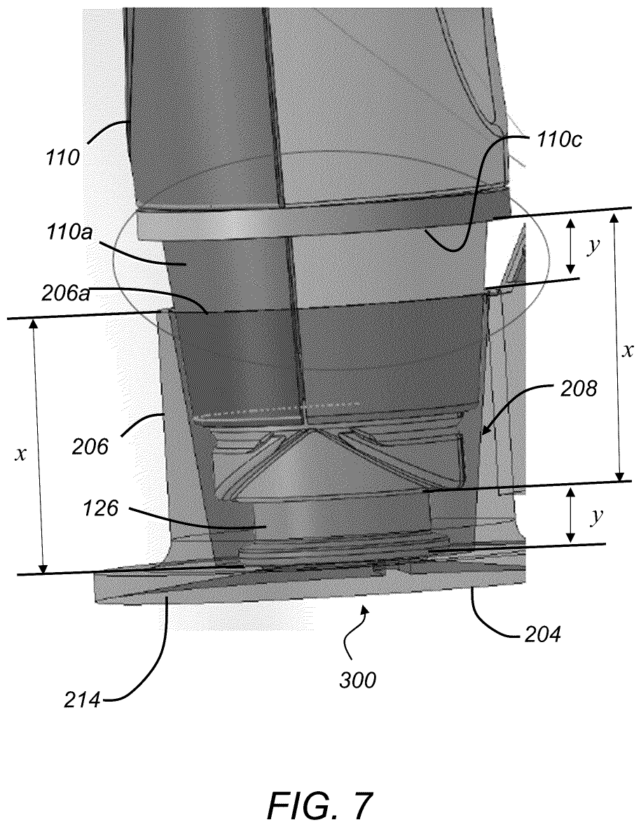

Referring to FIG. 7, an accessory 300 is shown according to another example. The accessory 300 can have a receptacle 206 and a base 214 configured as discussed above. Although not shown, the accessory 300 can optionally have at least one finger holder 218, such as a pair of finger holders 218, as discussed above. The accessory 300 and drug injection device 110 can be configured to together provide at least one visual indication of when the sliding sleeve 126 of the drug injection device 110 has reached the retracted position. For instance, the drug injection device 110 can comprise a first indicator 110c and the accessory 300 can comprise a second indicator 202, wherein alignment or mating of the first and second indicators 110c and 202 indicates that the sliding sleeve 126 has reached the retracted position. The indicators can be any suitable visual indicator, such as (without limitation) markings, lines, or mating surfaces.

FIG. 7 shows one example in which the first indicator of the drug injection device 110 is an edge (e.g., lip) 110c at the injection end 110a, and the second indicator of the accessory 300 is an edge 206a at the proximal end of the receptacle 206. The edge 110c contacts the edge 206a of the accessory 300 when the sliding sleeve 126 reaches the retracted position so as to give the user a visual indication that the sliding sleeve 126 is in the retracted position. The drug injection device 110 can have a dimension x from the edge 110c to a distal end of the housing 112 and/or to the skin-contacting surface 126a of the sliding sleeve 126 when the sliding sleeve 126 is in the retracted position. The cavity 208 can have a depth from the edge 206a of the receptacle 206 to the inner surface 210 of the receptacle 206 that is substantially equal to the dimension x. The dimension x is greater than a dimension y measured from the skin-contacting surface 126a of the sliding sleeve 126 to the distal end of the housing 112 when the sliding sleeve 126 is in the extended configuration.

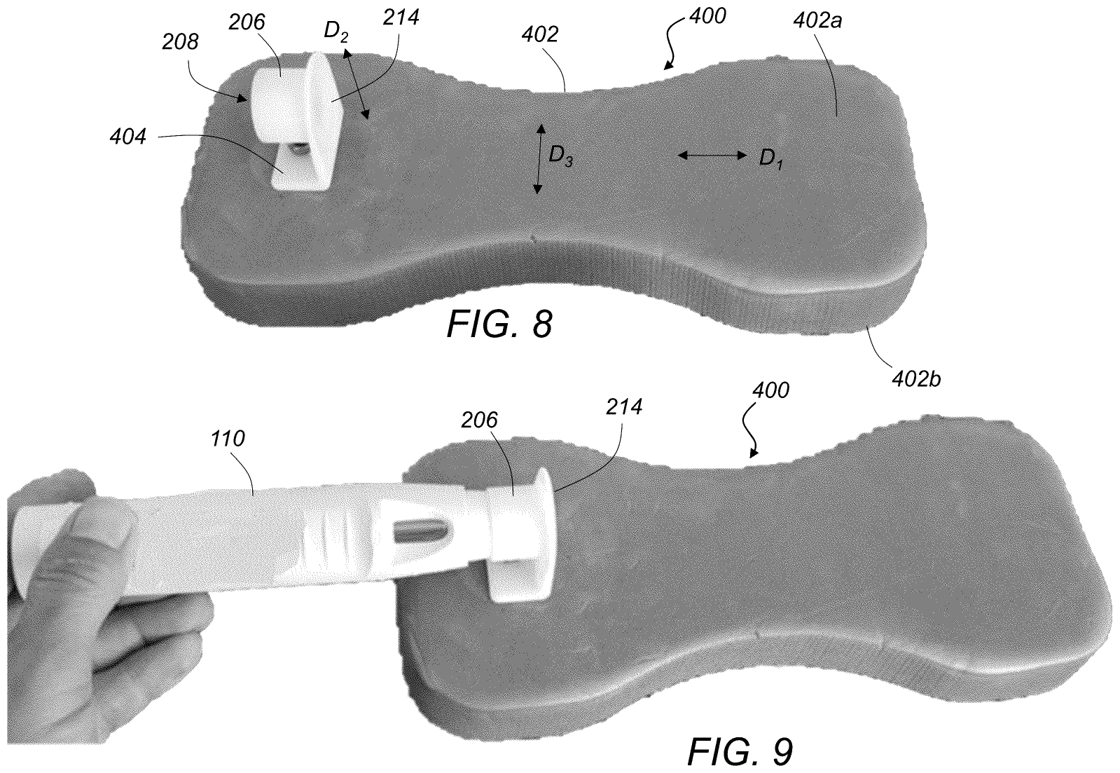

Turning now to FIGS. 8 to 10, an accessory 400 is shown according to yet another example. The accessory 400 comprises a receptacle 206 and a base 214 that can be configured as discussed above. The accessory 400 can optionally comprise an indicator or finger holder as discussed above. The accessory 400 can also comprise a positioning body 402, and an attachment member 404 that attaches the receptacle 206 and base 214 to the positioning body 402. The positioning body 402 is configured to orient the receptacle 206 and base 214 relative to the body of a patient so as to enable more consistent, repeatable, and reliable positioning of the injection device from one injection to the next. In at least one example, as shown in FIG. 10, the accessory 400 can be configured such that, when the positioning body 402 is placed adjacent a patient's arm pit, such that a first surface 402a faces the patient's upper arm and a second surface 402b faces the patient's torso, the receptacle 206 is in-line with the patient's triceps so as to position the drug injection device 110 to inject the drug into the triceps.

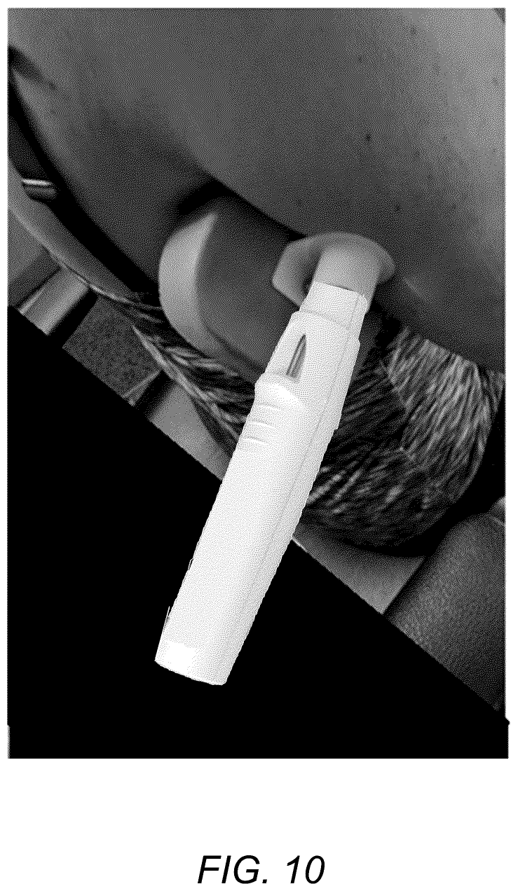

The positioning body 402 can have any suitable shape for positioning the base 214 against a desired injection site. For instance, in the example of FIGS. 8 and 9, at least a portion, up to an entirety, of the positioning body 402 can have a planar shape. The positioning body 402 can be a board or pad. The positioning body 402 can have a first surface 402a and a second surface 402b that are opposite one another. The first and second surfaces 402a and 402b can each have a cross-sectional dimension, such as a width and/or length, that is greater than a dimension from the first surface 402a to the second surface 402b. The attachment member 404 can be configured to attach the receptacle 206 and base 214 to one of the first and second surfaces 402a and 402b. In the example shown, the attachment member 404 comprises a plate 404 that is attached to the first surface 402a and to the base 214. The positioning body 402 extends along the first direction D1. In some examples, the positioning body 402 can be elongate along the first direction D1. The base 214 extends away from the first surface 402a along the second direction D3. The cavity 208 of the receptacle 206 has a central axis that extends along the first direction D1 and thus parallel to the first surface 402a of the positioning body 402. It will be understood that the receptacle 206 and base 214 can be attached to the positioning body 402 in any suitable manner and at any suitable location (e.g., on a side edge of the positioning body 402).

It should be noted that the illustrations and descriptions of the examples and embodiments shown in the figures are for exemplary purposes only, and should not be construed limiting the disclosure. One skilled in the art will appreciate that the present disclosure contemplates various embodiments. Additionally, it should be understood that the concepts described above with the above-described examples and embodiments may be employed alone or in combination with any of the other examples and embodiments described above. It should further be appreciated that the various alternative examples and embodiments described above with respect to one illustrated embodiment can apply to all examples and embodiments as described herein, unless otherwise indicated.

Unless explicitly stated otherwise, each numerical value and range should be interpreted as being approximate as if the word “about,” “approximately,” or “substantially” preceded the value or range. The terms “about,” “approximately,” and “substantially” can be understood as describing a range that is within 15 percent of a specified value unless otherwise stated.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

It will be understood that reference herein to “a” or “one” to describe a feature such as a component or step does not foreclose additional features or multiples of the feature. For instance, reference to a device having or defining “one” of a feature does not preclude the device from having or defining more than one of the feature, as long as the device has or defines at least one of the feature. Similarly, reference herein to “one of” a plurality of features does not foreclose the invention from including two or more, up to all, of the features. For instance, reference to a device having or defining “one of a protrusion and a recess” does not foreclose the device from having both the protrusion and the recess.

What is claimed:

1. An accessory for a drug injection device, the accessory comprising:
a proximal end and a distal end;
a receptacle defining:
a cavity that extends into the proximal end towards the distal end along a first direction and terminates at an inner surface, the cavity configured to receive an injection end of the drug injection device therein to position the drug injection device for delivery of a medication; and
an opening that extends into the distal end and through the inner surface, the opening configured to receive the needle of the drug injection device therethrough and having a cross-sectional dimension that is smaller than a cross-sectional dimension of the cavity;
a base at the distal end, the base comprising a skin-contacting surface that has a cross-sectional dimension that is greater than the cross-sectional dimension of the cavity; and
at least one finger holder that defines an opening configured to receive a finger of an operator of the drug injection device, the opening extending in a direction that is transverse to the first direction.

2. The accessory of claim 1, wherein the at least one finger holder comprises a pair of finger holders.

3. The accessory of claim 2, wherein the finger holders are disposed on opposing sides of the receptacle.

4. The accessory of any one of claims 1 to 3, wherein the at least one finger holder extends from the base along the first direction and then along a direction that is towards the receptacle.

5. The accessory of claim 4, wherein the at least one finger holder further extends back towards the base.

6. The accessory of any one of claims 1 to 5, wherein the at least one finger holder defines an at least partially closed shape around the opening.

7. The accessory of any one of claims 1 to 6, wherein the inner surface limits an insertion depth of the drug injection device into the receptacle.

8. The accessory of any one of claims 1 to 7, comprising a positioning body attached to the receptacle and base, the positioning body configured to orient the receptacle and base relative to a patient's body.

9. The accessory of claim 8, wherein the accessory is configured such that, when the positioning body is placed adjacent a patient's arm pit, between the patient's upper arm and the patient's torso, the receptacle is in-line with the patient's triceps so as to position the drug injection device to inject the drug into the triceps.

10. The accessory of any one of claims 8 and 9, wherein at least a portion of the positioning body has a planar shape.

11. The accessory of any one of claims 8 to 10, wherein the positioning body is a board or pad.

12. The accessory of any one of claims 8 to 11, wherein the positioning body has a first surface and a second surface that are opposite one another, and the first and second surfaces each have a cross-sectional dimension that is greater than a dimension from the first surface to the second surface.

13. The accessory of claim 12, wherein the accessory is configured to be positioned such that, when the first surface faces the patient's upper arm and the second surface faces the patient's torso, the receptacle is in-line with the patient's triceps so as to position the drug injection device to inject the drug into the triceps.

14. The accessory of claim 12, comprising an attachment member attaches the receptacle and base to one of the first and second surfaces.

15. The accessory of any of claims 8 to 14, wherein the positioning body is elongate along the first direction.

16. The accessory of any one of claims 8 to 15, wherein the base extends away from the positioning body along a second direction, transverse to the first direction.

17. The accessory of claim 1, comprising a first portion that includes at least a portion of the skin-contacting surface, and a second portion that supports the drug injection device, wherein the first portion is more flexible than the second portion so as to allow a user to flex the accessory to pinch a patient's skin during an injection.

18. A drug injection system, comprising:
the accessory of any of claims 1 to 17; and
the drug injection device.

19. The drug injection system of claim 18, wherein the drug injection device comprises:
a housing;
a syringe disposed within the housing, the syringe having a needle; and
a sliding sleeve that is configured to engage the inner surface of the accessory to move relative to the housing between an extended position, wherein the sliding sleeve extends from the housing, and a retracted position, wherein the sliding sleeve is retracted relative to the housing.

20. The drug injection system of claim 19, wherein the housing has a first indicator, the accessory has a second indicator, and the system is configured such that alignment or mating of the first and second indicators indicates that the sliding sleeve has reached the retracted position.

21. The drug injection system of claim 20, wherein the first indicator is a first edge, and the second indicator is a second edge at the proximal end of the receptacle that contacts the first edge when the sliding sleeve reaches the retracted position so as to give the user a visual indication that the sliding sleeve is in the retracted position.

22. The drug injection system of any one of claims 20 and 21, wherein the drug injection device has a dimension from the first indicator to a distal end of the housing or to a skin-contacting surface of the sliding sleeve when the sliding sleeve is in the retracted position, and the cavity has a depth from the second indicator of the receptacle to the inner surface that is substantially equal to the dimension.

23. A drug injection system, comprising:
a drug injection device, comprising:
a housing having a first indicator;
a syringe disposed within the housing, the syringe having a needle; and
a sliding sleeve that is configured to move relative to the housing between an extended position, wherein the sliding sleeve extends from the housing, and a retracted position, wherein the sliding sleeve is retracted relative to the housing; and
an accessory for a drug injection device, the accessory comprising:
a proximal end and a distal end;
a receptacle defining:
a cavity that extends into the proximal end towards the distal end along a first direction and terminates at an inner surface, the cavity configured to receive an injection end of the drug injection device therein such that the sliding sleeve engages the inner surface; and
an opening that extends through the inner surface to receive the needle of the drug injection device therethrough;
a base comprising a skin-contacting surface that has a cross-sectional dimension that is greater than a cross-sectional dimension of the sliding sleeve; and
a second indicator, wherein the system is configured such that alignment or mating of the first and second indicators indicates that the sliding sleeve has reached the retracted position.

24. The drug injection system of claim 23, wherein the first indicator is a first edge, and the second indicator is a second edge at the proximal end of the receptacle that contacts the first edge when the sliding sleeve reaches the retracted position so as to give the user a visual indication that the sliding sleeve is in the retracted position.

25. The drug injection system of any of claims 23 and 24, wherein the drug injection device has a dimension from the first indicator to a distal end of the housing or to a skin-contacting surface of the sliding sleeve when the sliding sleeve is in the retracted position, and the cavity has a depth from the second indicator of the receptacle to the inner surface that is substantially equal to the dimension.

26. An accessory for a drug injection device, the accessory comprising:
a proximal end and a distal end;
a receptacle defining:
a cavity that extends into the proximal end towards the distal end along a first direction and terminates at an inner surface, the cavity configured to receive an injection end of the drug injection device therein to position the drug injection device for delivery of a medication; and
an opening that extends into the distal end and through the inner surface, the opening configured to receive the needle of the drug injection device therethrough;
a base at the distal end, the base comprising a skin-contacting surface that has a cross-sectional dimension that is greater than a cross-sectional dimension of the cavity; and
a positioning body attached to the receptacle and base, the positioning body configured to orient the receptacle and base relative to a patient's body.

27. The accessory of claim 26, wherein the accessory is configured such that, when the positioning body is placed adjacent a patient's arm pit, between the patient's upper arm and the patient's torso, the receptacle is in-line with the patient's triceps so as to position the drug injection device to inject the drug into the triceps.

28. The accessory of any one of claims 26 and 27, wherein at least a portion of the positioning body has a planar shape.

29. The accessory of any one of claims 26 to 28, wherein the positioning body is a board or pad.

30. The accessory of any one of claims 26 to 29, wherein the positioning body has a first surface and a second surface that are opposite one another, and the first and second surfaces each have a cross-sectional dimension that is greater than a dimension from the first surface to the second surface.

31. The accessory of claim 30, wherein the accessory is configured to be positioned such that, when the first surface faces the patient's upper arm and the second surface faces the patient's torso, the receptacle is in-line with the patient's triceps so as to position the drug injection device to inject the drug into the triceps.

32. The accessory of claim 30, comprising an attachment member attaches the receptacle and base to one of the first and second surfaces.

33. The accessory of any of claims 26 to 32, wherein the positioning body is elongate along the first direction.

34. The accessory of any one of claims 26 to 33, wherein the base extends away from the positioning body along a second direction, transverse to the first direction.
